(19) United States
(12) Patent Application Publication
(10) Pub. No.: US 2025/0261914 A1
(43) Pub. Date: Aug. 21, 2025

(54) MAGNETIC RESONANCE IMAGING COMPATIBLE RAPID DYNAMIC SUPPORT CONTROL SYSTEM

(71) Applicants: The Chinese University of Hong Kong, Hong Kong (CN); The Hong Kong Polytechnic University, Hong Kong (CN)

(72) Inventors: Kai-yu TONG, Hong Kong (CN); Zhongping YE, Hong Kong (CN); Shuk Fan TONG, Hong Kong (CN); Yiu Wan YIP, Hong Kong (CN); Qiwen LEI, Hong Kong (CN); Jingyi MA, Hong Kong (CN)

(21) Appl. No.: 18/901,085

(22) Filed: Sep. 30, 2024

(30) Foreign Application Priority Data
Feb. 21, 2024 (CN) 202410193257.7

(51) Int. Cl. A61B 6/04; A61B 5/055

(52) U.S. Cl. CPC A61B 6/0407; A61B 5/055

(57) ABSTRACT

A rapid dynamic support control system compatible with magnetic resonance imaging (MRI) is configured for providing corrective pressure to non-freely moveable human joints during MRI scanning to achieve a prompt and targeted posture adjustment. The system includes a dynamic composite pressure support device with MRI imaging contrast-enhancing properties. The support device includes multiple layers that include a sealing layer and an MRI-visible layer. A pressure adjustment device with an air pump and air valves regulates the support device's pressure. A control system monitors actuator pressure and dynamically adjusts pressure distribution among multiple support devices in real-time. During MRI scanning, patients wear the support device inside the MRI scanner and adjust it using a pressure adjustment circuit connected through MRI-compatible material conduits. Rapid and precise support pressure adjustments are offered in the fields of medical imaging and treatment, thereby optimizing treatment outcomes and patient comfort.

### CROSS-REFERENCE TO RELATED APPLICATION

This application claims the benefit of Chinese Application No. 202410193257.7, filed Feb. 21, 2024, which is hereby incorporated by reference in its entirety.

### FIELD OF THE INVENTION

The present invention relates to the technical field of medical devices, specifically to an MRI compatible rapid dynamic support control system.

### BACKGROUND

Providing patients with posture correction or limb fixation often requires robust external support. For instance, scoliosis patients often wear posture correction braces to mitigate scoliosis progression, while stroke patients may require wearable assistive devices to enhance movement in stiff limbs. However, assessing the biomechanical condition of target bones or muscles in real-time poses challenges, hindering therapists' ability to adjust assistive devices based on immediate effects to optimize correction outcomes or corrective action. Therefore, the ability to provide rapid dynamic support adjustment and promptly respond to support device effects is crucial for delivering effective rehabilitation treatment to patients.

In recent years, several technological solutions have emerged to address these challenges. For example, the US patent U.S. Pat. No. 10,639,185B2 describes a spinal treatment device, system, and method that utilizes multiple linear actuators to interact with a brace. This enables selective motion control and feedback with multiple degrees of freedom, allowing for active training or passive support. The device can also estimate muscle strength in the user. The support solution offers personalized adjustment strategies and allows for quick adjustments. However, the complex execution mechanism adds additional weight and is more time consuming to be adjusted for different situations on patients. The specific effectiveness of the device can only be indirectly inferred.

The US patent U.S. Pat. No. 8,146,599B2 discloses a system for positioning patient on a treatment table that comprises a patient support panel and positioning component to immobilize a portion of patient's body in a predetermined fixed position. The system allows fixation of a patient's body where needed to be immobilized and enables the transfer of patients to an imaging device. However, the adjustment of the fixation of the patient needs to be carried out by the therapist and the positioning devices. It is difficult to be applied quickly before the medical imaging.

MRI is a non-invasive, radiation-free, high-resolution medical imaging technology. Unlike X-ray-based imaging techniques such as X-ray imaging and computed tomography scanning, MRI offers superior spatial imaging without radiation exposure, showcasing soft tissue structure and MRI-visible materials within the scanning area. With latest scanning sequence, the image of bone and cartilage tissues can be imaged. For subjects requiring bone fixation or correction evaluation, MRI provides detailed anatomical data to assess potential impacts and allows for multiple scans in a short period without radiation effects accumulation. The posture can be adjusted in a short period of time by adjusting different pressure and support device positions.

Traditional structural support systems with metal structure are incompatible with strong magnetic fields and radiofrequency magnetic fields present in MRI environments, posing harm risks to subjects. Conventional pressure support actuators, such as traditional airbags, lack contrast materials, making it challenging to determine the shape of the orthopedic actuator after being driven and its structural deformation impact on human body surface.

### SUMMARY

The subject invention presents a Magnetic Resonance Imaging Compatible Rapid Dynamic Support Control System, targeting the field of medical devices and support the design of orthotic devices. It addresses the need for effective posture correction and limb fixation by providing real-time adjustable external support, patients with conditions such as scoliosis, brain injury or stroke. Traditional solutions often lack fast evaluation and adjustment capabilities, hindering optimal outcomes. The system leverages MRI technology, offering non-invasive, radiation-free imaging to assess different body tissue, hard tissue and soft tissue accurately. Unlike conventional systems, it ensures compatibility with MRI environments, enabling precise support adjustments and immediate response to clinical needs. By integrating dynamic composite pressure support devices with MRI-visible materials and advanced control systems, the invention facilitates rapid and targeted posture adjustments, optimizing treatment outcomes in medical imaging and rehabilitation.

The present invention addresses the technical challenge of dynamically observing and adjusting support pressure on non-freely flexible joints during medical procedures, a longstanding issue in orthopedic treatment. Conventional braces and assistive devices lack real-time monitoring capabilities, impeding rapid and targeted adjustments essential for optimizing treatment outcomes. To overcome these limitations, the present invention introduces a Magnetic Resonance-Compatible Rapid Dynamic Support Control System, revolutionizing the field of medical imaging and orthopedic care.

A technical solution offered by the present invention is a comprehensive system designed to achieve swift and precise adjustment of support pressure on various stiff joints through the innovative integration of dynamic composite pressure support structures visible under MRI scanning, combined with a sophisticated pressure adjustment apparatus. This system ensures the dynamic stability of support pressure applied to the patient's body, making it particularly suitable for treating stiff joints such as cervical, thoracic, lumbar, upper and lower limbs, neck, and head.

Embodiments of the subject invention include dynamic composite pressure support, comprising composite pressure actuators specifically engineered to be visible under MR scanning. These actuators, customizable for diverse applications, are constructed with multiple layers including a sealing layer for pressure containment and an MRI visible layer containing hydrogen-rich materials for deformity visualization. The incorporation of external fabric wrapping enhances comfort and minimizes direct skin contact, ensuring patient compliance and comfort during treatment.

A pressure adjustment device is configured to facilitate precise pressure adjustment for each actuator, featuring a pressure regulation system and a fast responsive control system. Embodiments of this system operate by pre-pressurizing gas within pressure vessel via an advanced air pump mechanism, with pressure distribution controlled by gas valves and continuously monitored in real-time. The control system, comprising a highly adaptable controller and associated circuits, dynamically regulates pressure based on sensor feedback and external input parameters, ensuring optimal treatment outcomes.

In certain embodiments, during MRI scanning, only the composite pressure support is positioned within the scanning room, connected to the pressure adjustment circuit via an MRI-compatible connection tube. Therapists can efficiently coordinate pressure adjustments with MR scanning results to swiftly optimize joint posture, leveraging monitoring and real-time adjustment capabilities to enhance treatment precision and patient comfort.

In conclusion, the subject invention provides a system and a method for medical imaging and orthopedic treatment. By enabling real-time adjustment of support pressure for stiff joints, this magnetic resonance compatible rapid dynamic support control system offers a solution with broad applications in medical practice.

### BRIEF DESCRIPTION OF DRAWINGS

Embodiments will hereinafter be described with reference to the accompanying drawings, which have not necessarily been drawn to scale. Specific structural and functional details depicted in the drawings are provided for illustrative purposes and may vary in actual implementations. Furthermore, elements in the drawings may be simplified for clarity and understanding. Throughout the drawings, like reference numerals denote similar elements or components, facilitating ease of comparison and comprehension. The following detailed description, in conjunction with the accompanying drawings, is intended to elucidate various embodiments of the invention and should not be interpreted as limiting the scope of the invention to the precise embodiments disclosed herein.

FIG. 1: Schematic diagram illustrating the implementation of rapid adjustment by the magnetic resonance compatible rapid dynamic support control system within a magnetic resonance scanning room with a pressure vessel.

FIG. 2: Schematic diagram demonstrating the adjustment process by the magnetic resonance compatible rapid dynamic support control system within a magnetic resonance scanning room without a pressure vessel.

FIG. 3: Example schematic diagram depicting the strategy employed by the magnetic resonance compatible rapid dynamic support control system in conjunction with magnetic resonance 5 scanning.

FIG. 4: Schematic diagram illustrating the basic structure of the composite pressure actuator imaged by magnetic resonance scanning.

FIG. 5: Schematic diagram showcasing the sectional structure of the composite pressure actuator imaged by magnetic resonance scanning.

FIG. 6: Schematic diagram depicting the wearing effect of the dynamic composite pressure support on the user, along with X-ray results.

FIG. 7: Schematic diagram illustrating the application of pushing orthopedic force to spinal joints by the dynamic composite pressure support.

FIG. 8: Schematic diagram showcasing the imaging and orthopedic adjustment of the magnetic resonance imaging composite pressure actuator during magnetic resonance scanning.

FIG. 9: Example schematic of a scanning illustration demonstrating the relative positional changes of the dynamic composite pressure support device on the user's body due to adjustments.

FIG. 10: Explanatory schematic diagram the relative positional changes of the dynamic composite pressure support device on the user's body due to adjustments.

FIG. 11: Schematic diagram illustrating the correction of orthopedic relationship by coordinating multiple airbags on the user's body with the dynamic composite pressure support.

FIG. 12: Schematic diagram demonstrating the push-type structure and action force of the magnetic resonance imaging composite pressure actuator.

FIG. 13: Schematic diagram illustrating the folding deformation of the extension structure and bending structure of the magnetic resonance imaging composite pressure actuator.

FIG. 14: Schematic diagram illustrating the force application effect of the extended structure of the composite pressure actuator.

FIG. 15: Schematic diagram illustrating the force application effect of the bending structure of the composite pressure actuator.

FIG. 16: Schematic diagram showcasing the wearing deformation effect of the extension structure and bending structure of the magnetic resonance imaging composite pressure actuator on the user.

FIG. 17: Typical use case schematic diagram of the push-type structure of the magnetic resonance imaging composite pressure actuator during magnetic resonance scanning.

FIG. 18: Typical use case schematic diagram of the extension structure of the magnetic resonance imaging composite pressure actuator during magnetic resonance scanning.

FIG. 19: Typical use case schematic diagram of the bending structure of the magnetic resonance imaging composite pressure actuator during magnetic resonance scanning.

FIG. 20: Logical relationship flowchart of the actuator pressure adjustment of the magnetic resonance compatible rapid dynamic support control system.

FIG. 21: Logical relationship flowchart of the pressure vessel pressure adjustment of the magnetic resonance compatible rapid dynamic support control system.

FIG. 22: Logical relationship flowchart of pump pressurization adjustment of the magnetic resonance compatible rapid dynamic support control system without a pressure vessel.

FIG. 23: Logical relationship flowchart of the actuator pressure maintenance of the magnetic resonance compatible rapid dynamic support control system.

FIG. 24: Example schematic diagram demonstrating the actual pressure adjustment effect of the actuator of the magnetic resonance compatible rapid dynamic support control system.

FIG. 25: Example schematic diagram showcasing the actuator pressure maintenance and pressure change of the pressure container of the magnetic resonance compatible rapid dynamic support control system.

FIG. 26: Pressure curve schematic diagram of the actuator adjustment without a pressure vessel in the magnetic resonance compatible rapid dynamic support control system.

FIG. 27: Pressure curve schematic diagram of the actuator adjustment with a pressure vessel in the magnetic resonance compatible rapid dynamic support control system.

FIG. 28: Example schematic diagram of the pressure fluctuations caused by changes in external loads on the actuator and the processed fluctuations.

### DETAILED DESCRIPTION

Embodiments of the present invention will be described below with reference to the accompanying drawings, wherein like reference numerals refer to like elements, and various specific embodiments are provided for illustrative purposes only and are not intended to limit the scope of the invention.

The present invention relates to an MRI-compatible rapid dynamic support control system designed to address technical issues associated with conventional support devices and auxiliary instruments, such as difficulties in observing the actual effects of support pressure on targeted areas of a patient's body, challenges in visualizing the contact between support device and the patient, and the need for rapid and targeted adjustments.

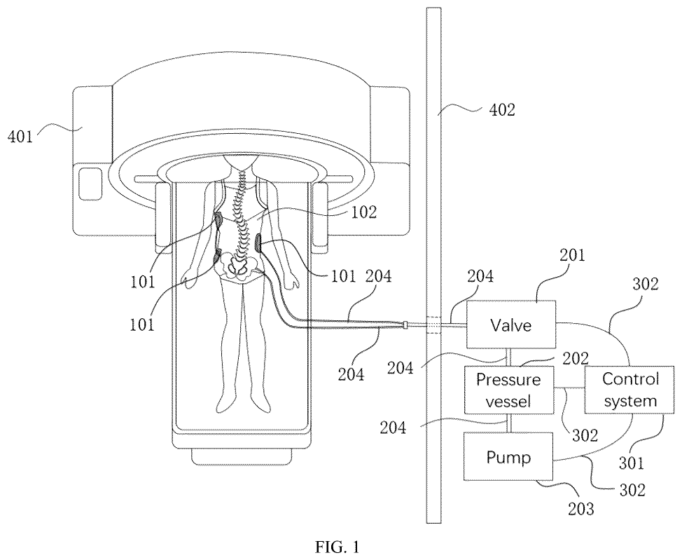

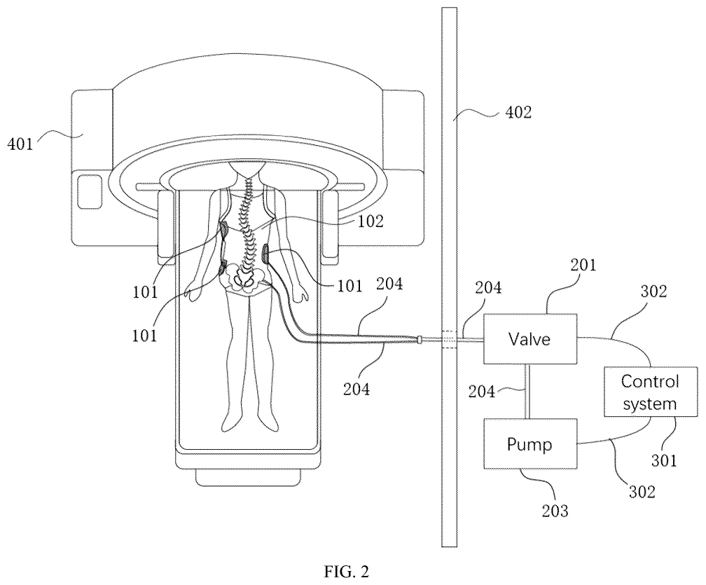

In a specific embodiment shown in FIG. 1, the system operates within an MRI scanning room, where the system is coordinated with an MRI scanner 401 to address the corrective pressure needs of a patient's joints. The patient wears a dynamic composite pressure support brace 102 and places a composite pressure actuator 101, visible under MRI scanning, at the site where pressure is applied. In the implementation of the present invention, to achieve rapid and accurate support pressure adjustment, the dynamic composite pressure support device includes at least one composite pressure actuator 101 visible under MRI scanning. It may optionally be combined with an MRI-compatible brace 102, which can be made of MRI-compatible materials such as soft materials (e.g., pure cotton textiles) or rigid materials (e.g., plastic). The design of the brace 102 can be optimized for treatment effects by varying its shape according to different usage positions. During MRI scanning, only the composite pressure actuator 101 within the composite pressure support device and the brace 102 are placed inside the scanning room with the MRI scanner, while the pressure adjustment device is positioned outside the scanning room and connected to the actuator 101 and brace 102 through an MRI-compatible material conduit 204 passing through the MRI scanning room wall 402. The pressure adjustment circuit includes a pressure valve 201, pressure vessel 202, and air pump 203, which are controlled by a control system 301 to regulate the pressure applied to the actuator. The control system 301 also collects pressure data from within the actuator 101 through the conduit 204 and transmits it via a data line 302. As the air pump 203 can pre-pressurize the pressure vessel 202, during the adjustment process in coordination with MRI scanning, the pressure within the vessel 202 can be rapidly transmitted to the actuator 101, enabling rapid pressure adjustment. In a system without a pressure vessel, as shown in FIG. 2, the system can still apply pressure to the actuator 101 by driving the air pump 203, but the response speed is limited by the performance of the air pump.

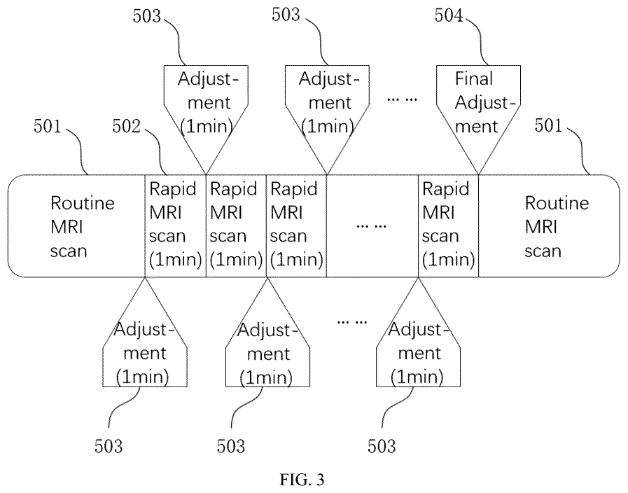

In practical use, in an embodiment and as depicted in FIG. 3, a typical workflow involves analyzing results from routine MRI scans 501 and multiple rapid MRI scans 502, which takes about 1 minute, followed by rapid adjustments 503 using the MRI-compatible rapid dynamic support control system to quickly identify the pressure distribution between the actuators 101 for the patient's condition, optimizing the required pressure and location within 1 minute. After the final adjustment 504, another routine MRI scan will be performed to record the immediate effect of joint correction with high resolution.

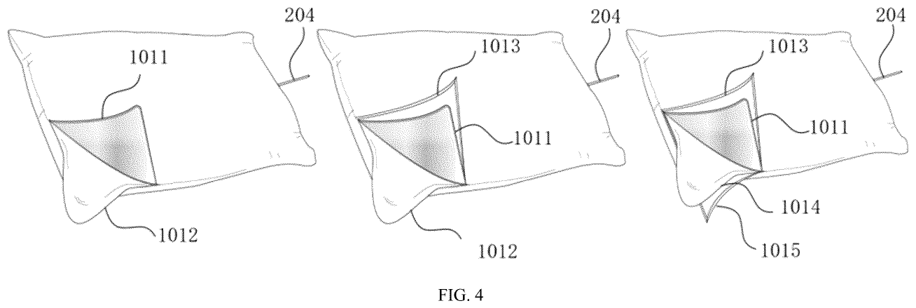

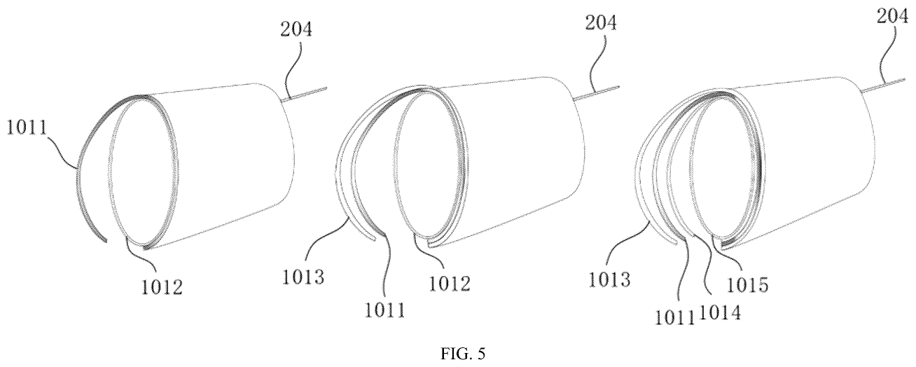

The composite pressure actuators 101, which are visible under MRI, are constructed from multi-layer composite materials, in embodiments as shown in FIGS. 4 and 5, comprising a sealing layer 1012 and an MRI-visible layer 1011. The sealing layer 1012 withstands gas pressure, provides the required support or orthopedic pressure, and maintains the support shape. The MRI-visible layer 1011 comprises materials rich in hydrogen atoms, such as hydrogels, fish oils, etc., to reflect the deformation of the actuators under MRI. By controlling the concentration of hydrogen atoms in the MRI-visible layer 1011 material, interference from artifacts on the support effect can be minimized. To improve comfort and isolate direct contact between the actuator surface and the skin, fabric 103 may be used to wrap the actuator externally.

Specifically, the sealing layer 1012 may be made of composite materials such as TPU nylon composite, and may be divided into a gas storage layer 1014 made of materials with gas sealing properties, such as TPU, and a deformation constraint layer 1013 made of materials with deformation constraint properties, such as nylon. The design of the sealing layer 1012 ensures that the actuator 101 deforms as intended and provides sufficient pressure for support.

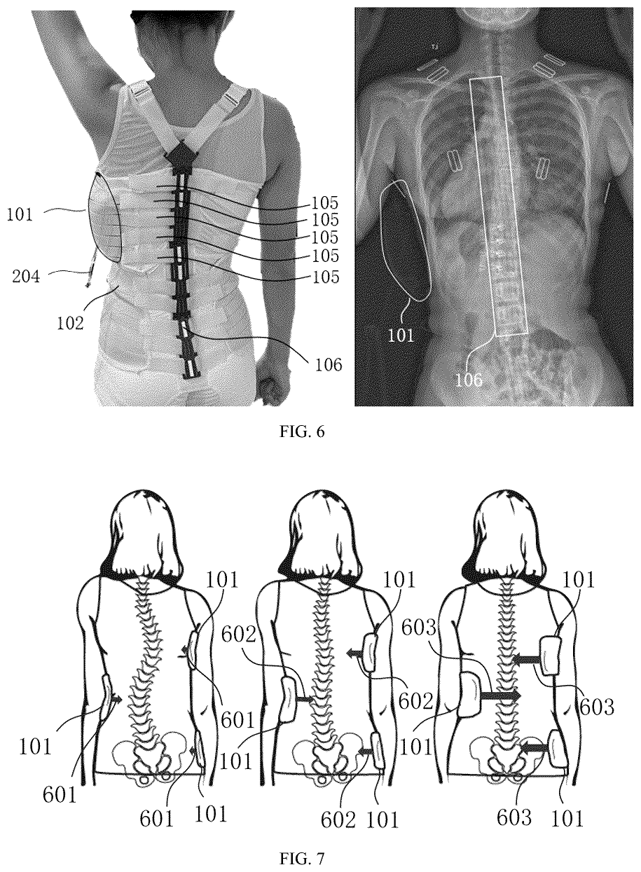

In typical scenarios, such as spinal joint orthopedics, the actual wearing mode, as shown in FIG. 6, involves wearing the support brace 102 and placing the actuator 101 inside to correct spinal posture. The actuator 101 is connected to the pressure control system via an air pipe 204. Various MRI-compatible materials may be used on the frame to assist the actuator in providing orthopedic pressure, such as elastic straps 105 to provide lateral support and an MRI-compatible skeleton 106 to stabilize the structure of the frame 102 and provide support points for the straps 105.

When applying orthopedic force to spinal joints, as shown in FIG. 7, the effects of the push-type orthopedic force are demonstrated. The actuator 101 can generate different orthopedic forces by adjusting the pressure, including the basic orthopedic force 601 generated by external supports and the material itself, the gradually increasing orthopedic force 602 as the air pressure increases, and the maximum orthopedic force 603 under maximum deformation and maximum tolerable air pressure.

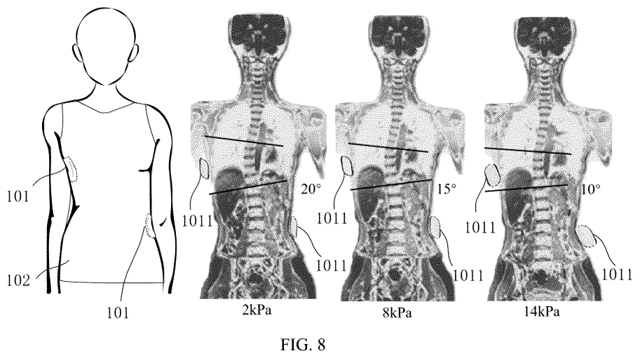

In actual use, as demonstrated in FIG. 8, the MRI scan results and the adjustment effects are shown. Inside the MRI scanning room, the patient wears the MRI-compatible brace 102 and the MRI-visible actuator 101 corrects the spine. In this example, after the MRI scan, the patient's spine joint morphology and related tissue conditions, as well as the morphology of the MRI-visible layer 1011 of the actuator, can be displayed. By analyzing the joint morphology, the actuator can be driven to gradually increase pressure to achieve straightening of relatively stiff spinal joints, while simultaneously observing other tissue states and actuator morphology to assist therapists in treatment judgments and situation analysis.

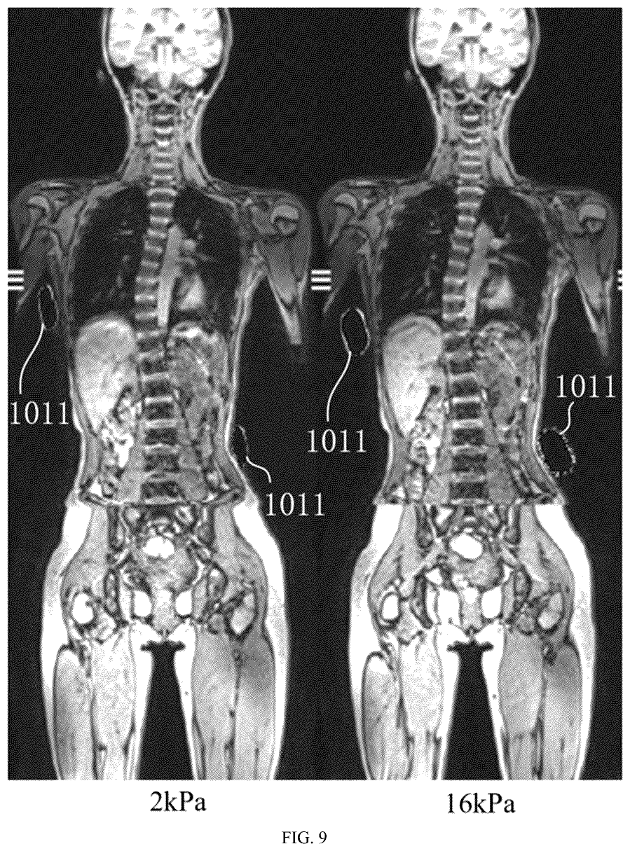

As shown in FIG. 9, the dynamic composite pressure support may cause relative position changes on the user. MRI scan images show that when the actuator 101 is not pressurized, its visible layer 1011 position is at the highest point of spinal curvature. As the correction progresses, the pressurized actuator expands, causing positional offsets and affecting the direction of corrective force application.

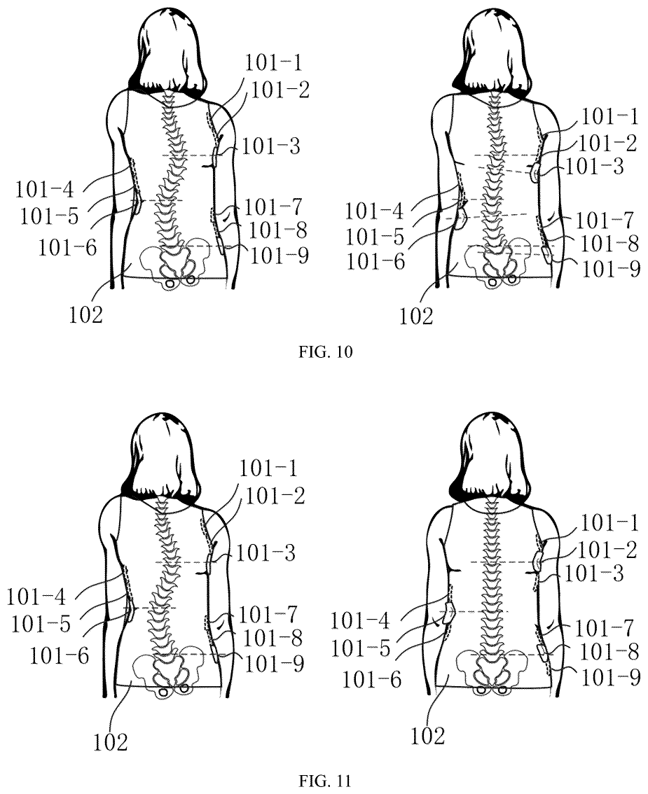

This offset phenomenon is further illustrated in the schematic diagram of FIG. 10. In the initial unpressurized state, the heights of the actuators 101-3, 101-6, and 101-9 are aligned with the maximum curvature position of the spine. However, as the actuators expand, the position of actuator 101-3 shifts relative to the position of maximum spinal curvature. Additionally, multiple actuators 100-1, 101-2, 101-4, 101-5, 101-7, 101-9 are placed at different locations along the trunk within the frame 102.

To address this positional offset during correction, adjustments can be made by coordinating multiple airbags, as shown in FIG. 11. By shifting to actuators with higher relative positions, such as actuators 101-2, 101-5, 101-8, the relative positional offset can be corrected, ensuring the orthopedic correction force is applied at the desired location.

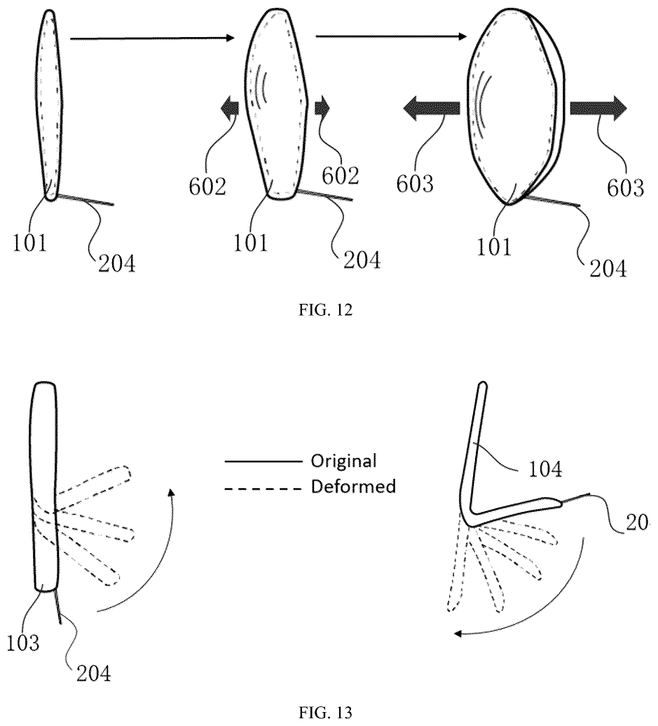

In catering to diverse therapeutic needs, actuators can apply varying corrective forces. A typical form of actuator is the compression-type orthosis 101, as illustrated in FIG. 12. This compression-type orthosis 101 operates through air pressure for both pressurization and depressurization. As pressure increases, the corrective force 602 gradually escalates until reaching maximum deformation and maximum pressure corrective force 603.

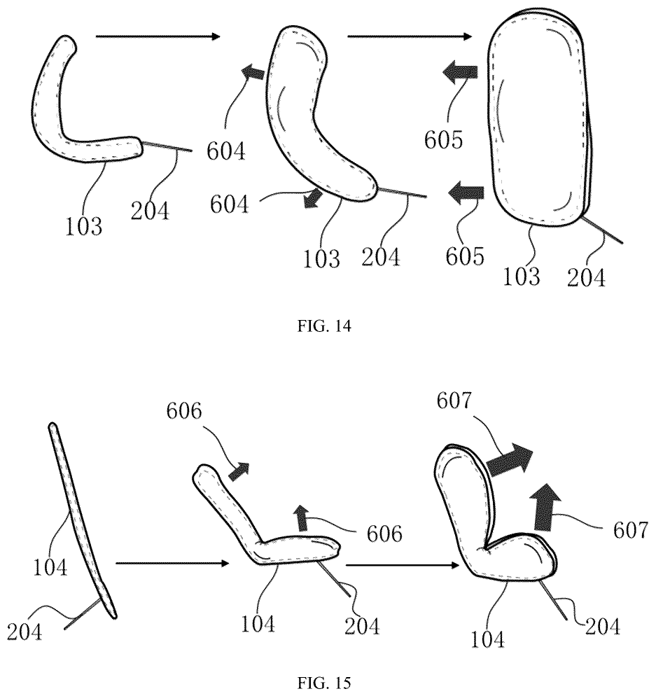

Apart from the compression-type orthosis 101, other typical forms of actuators are depicted in FIG. 13. The extension-type actuator 103 can be bent to any angle when pressurized and gradually straightened when depressurized. Under such adjustments, these actuators are worn in a relatively soft state by the user. The effect of pressurizing the folded extension-type actuator 103 to apply external force is illustrated in FIG. 14. As pressure increases, the folded extension-type orthosis 103 gradually straightens, providing force 604 to gradually extend the joints until reaching maximum deformation and maximum pressure corrective force 605. Similarly, the effect of pressurizing the straightened extension-type actuator 103 to apply external force is depicted in FIG. 15. As pressure increases, the straightened extension-type orthosis 103 gradually straightens further, providing force 606 to gradually bend the joints until reaching maximum deformation and maximum pressure corrective force 606.

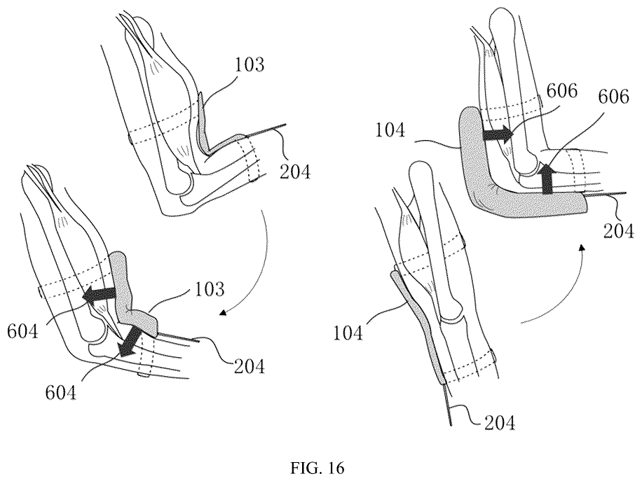

The effects of wearing the extension-type actuator 103 and the bending-type actuator 104 at the joints are shown in FIG. 16. The extension-type actuator 103, by increasing pressure, can enhance straightening force 604 to gradually straighten the joints. The bending-type actuator 104, by increasing pressure, can enhance bending force 606 to gradually achieve a bent state in the joints.

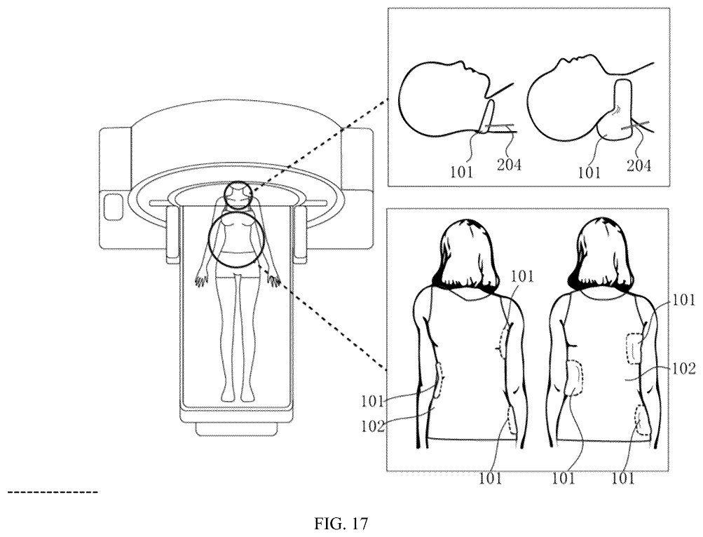

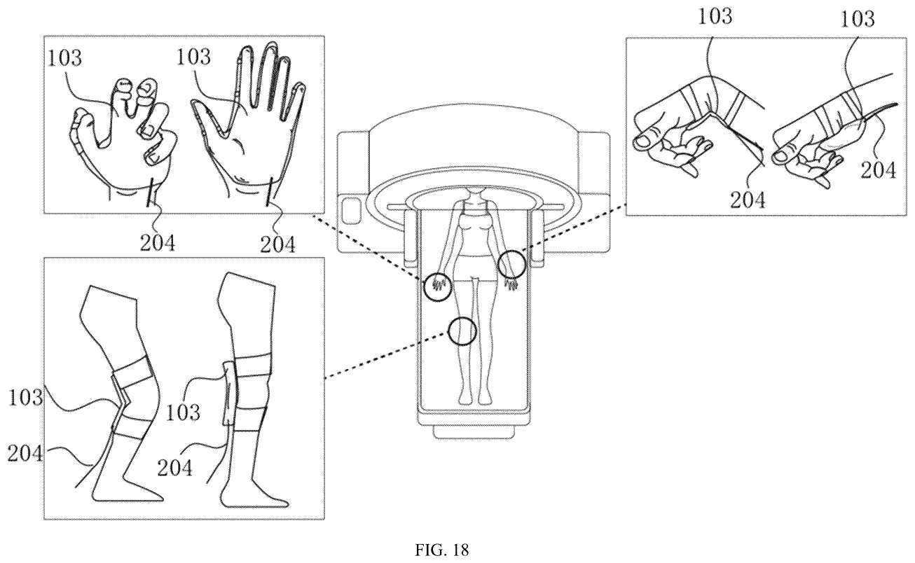

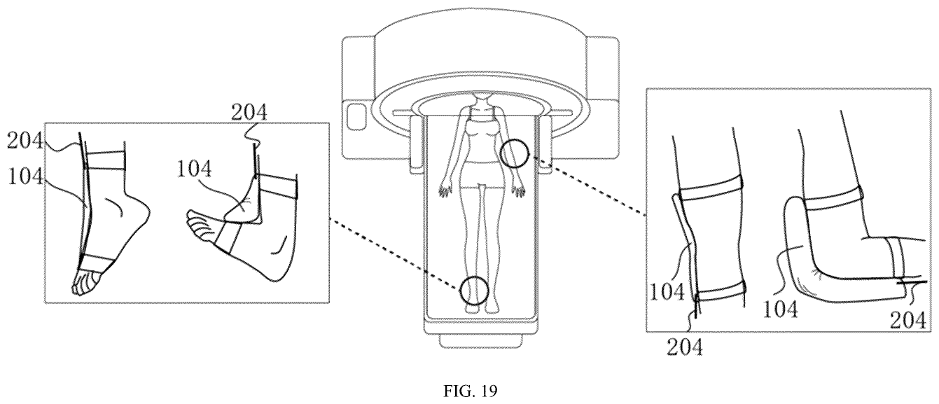

When addressing various usage scenarios, therapists can employ different actuators based on requirements. Here, only typical usage scenarios are outlined. A typical scenario for the compression-type actuator 101 is depicted in FIG. 17, where it can alter neck posture through pipeline 204 or adjust the user's spinal posture via pressurization. A typical scenario for the extension-type actuator 103 is shown in FIG. 18, where it can be crafted into various forms and worn on palms, wrists, knees, etc., applying pressure to extend them. A typical scenario for the bending-type actuator 104 is illustrated in FIG. 19, where it can be worn on ankles and elbow joints, applying pressure to induce bending. The aforementioned typical usage scenarios serve as illustrative examples of different actuator applications. Users can expand their usage methods by modifying actuator forms and wearing positions.

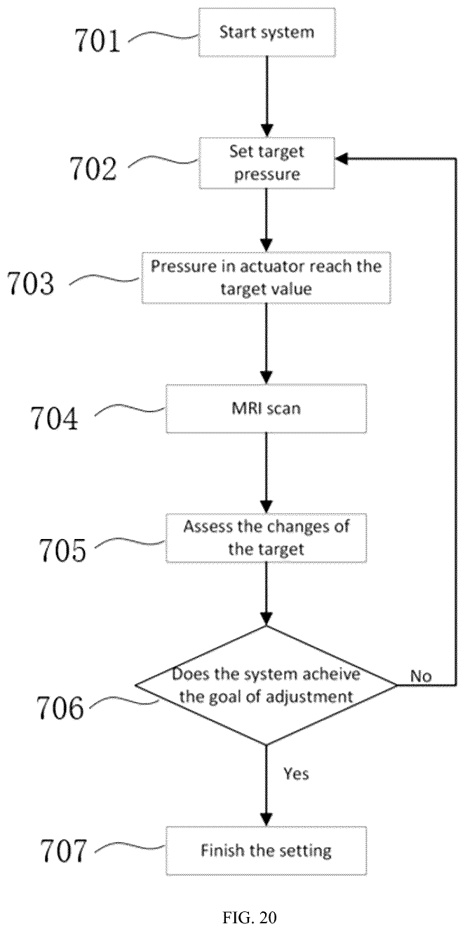

FIG. 20 depicts the flowchart of the pressure adjustment logic for the dynamic support system during usage of the MRI-compatible fast dynamic support control system. The specific pressure adjustment logic is outlined as follows:
701: After arranging and performing safety checks within the MRI scanning room, initiate the equipment. The dynamic support device pressurizes the pressure vessel to prepare for providing the required pressure to the actuators.
702: Therapists set the pressure magnitude for each actuator according to treatment and adjustment goals.
703: Upon completing pressure settings, wait for the actuators to reach and stabilize at the target pressure.
704: Once pressure stabilization is achieved, initiate MRI scanning and obtain scan results.
705: Therapists use MRI scans to assess real-time posture of the target body parts, including changes in target muscles or bones, actuator status, and effects on surrounding tissues.
706: Evaluate whether adjustments to relative joints meet the target based on MRI scan results. If not, redesign the target pressure of the pressure support device.
707: If adjustments meet the target, complete support settings.

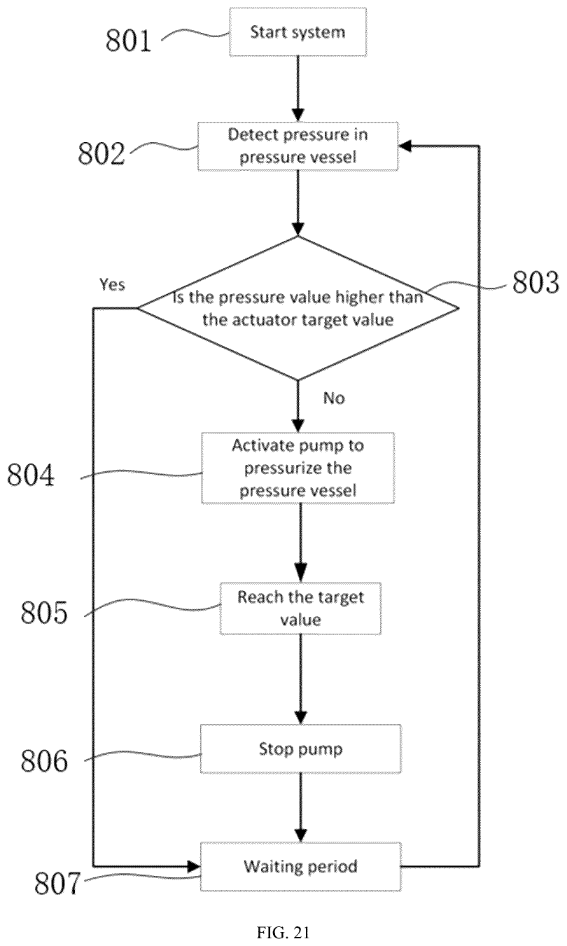

FIG. 21 depicts the flowchart of the logic for adjusting pressure within the pressure vessel of the MRI-compatible fast dynamic support control system. The specific pressure adjustment logic is as follows:
801: After arranging and performing safety checks within the MRI scanning room, initiate the equipment, and the system conducts self-checks for all functional modules to prepare for use.
802: The control system detects the current pressure within the pressure vessel.
803: Sensor readings determine whether the pressure within the pressure vessel meets the condition of being greater than the upper limit of all actuator target pressures. If so, wait for the system's designated wait time.
804: If not, the pump pressurizes the pressure vessel.
805: The control system continuously monitors until the pressure within the pressure vessel reaches the predetermined pressure value set by the program.
806: The control system stops the pump operation.
807: After the default wait duration, recheck the pressure within the pressure vessel.

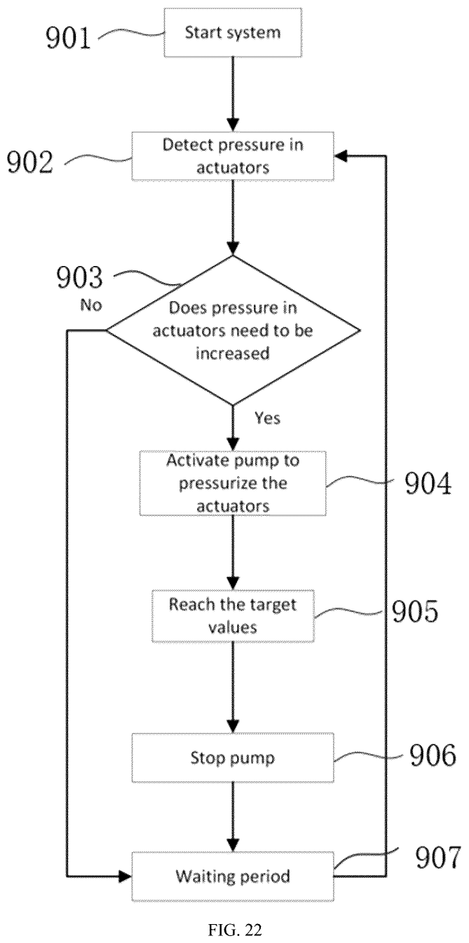

FIG. 22 depicts the flowchart of the logic for adjusting pressure via pump without a pressure vessel in the MRI-compatible fast dynamic support control system. The specific operating logic is as follows:
901: After arranging and performing safety checks within the MRI scanning room, initiate the equipment, and the system conducts self-checks for all functional modules to prepare for use.
902: The control system detects the current pressure within the actuator.
903: Sensor readings determine whether the pressure within the actuator meets the target pressure condition. If so, wait for the system's designated wait time.
904: If not, the pump is directly activated to pressurize the actuator.
905: The control system continuously monitors until the pressure within the actuator reaches the predetermined pressure value set by the program.
906: The control system stops the pump operation.
907: After the default wait duration, recheck the pressure within the actuator.

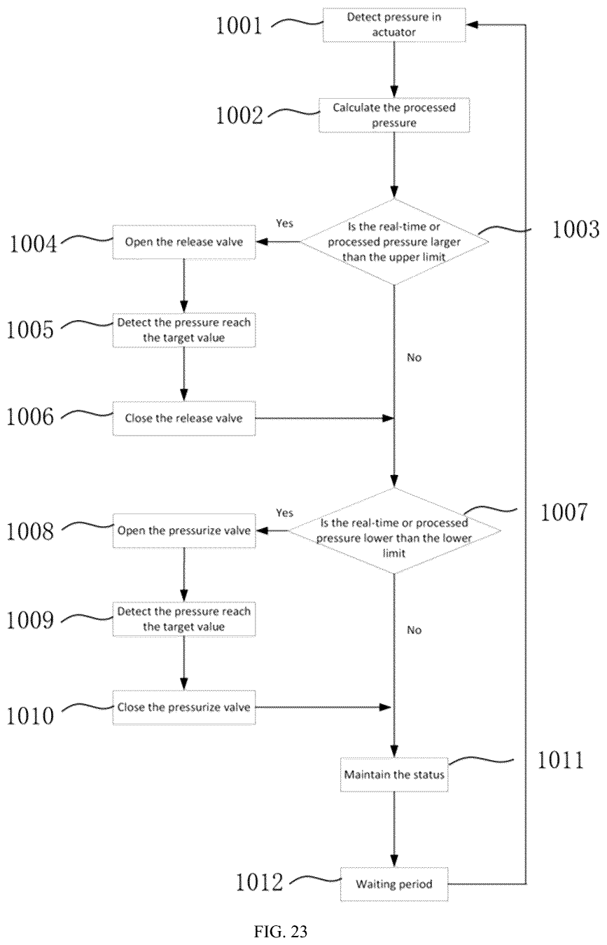

FIG. 23 depicts the flowchart of the logic for adjusting pressure in the composite pressure actuator via MRI scanning. Through this control logic, the control system within the pressure adjustment device performs corresponding detection and calculations, adjusting the pressure within the composite pressure support device through control of the air pump 203 and air valve 201 to maintain dynamic equilibrium with the target pressure. The specific control logic for maintaining dynamic pressure stability by the control system 301 is as follows:
1001: Detect the pressure within the actuator via pressure sensor.
1002: Calculate the average pressure over a set duration based on the latest pressure value, obtaining a processed dynamic pressure value.
1003: Determine whether the real-time or dynamic processed pressure value exceeds the respective set upper limits.
1004: If it exceeds the upper limit, open the relief valve corresponding to the actuator.
1005: Monitor the pressure within the actuator until it reaches the set pressure value.
1006: Close the relief valve corresponding to the actuator.
1007: If it does not exceed the upper limit, determine whether the real-time or dynamic processed pressure value is below the respective set lower limits.
1008: If it is below the lower limit, open the inflation valve corresponding to the actuator.
1009: Monitor the pressure within the actuator until it reaches the set pressure value.
1010: Close the inflation valve corresponding to the actuator.
1011: If it is not below the lower limit, maintain the current state of the actuator.
1012: After the default wait duration, recheck the pressure within the actuator.

The relevant control parameters can be adjusted through connection with external computing devices, receiving instructions, and making adjustments. The connection method can be wired or wireless.

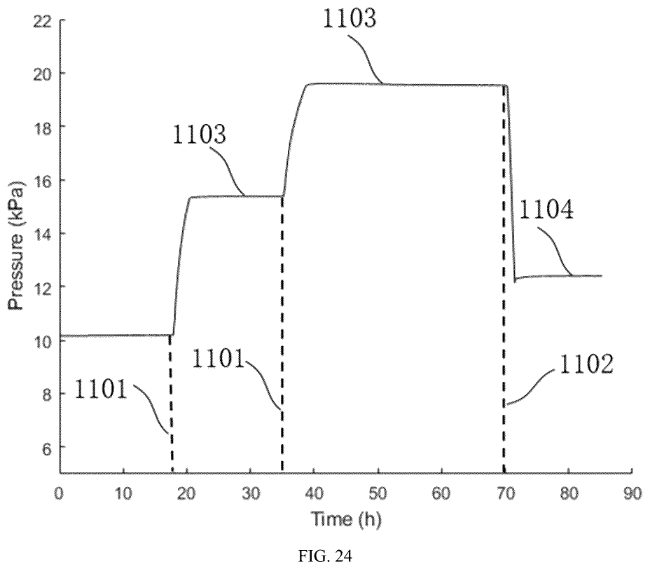

FIG. 24 illustrates the control system's pressure adjustment for the actuator. When the pressure adjustment command 1101 is received, the control system initiates pressurization of the target actuator. By controlling the corresponding air valve switch, the air pressure is increased until reaching the target air pressure 1103. It can also accept depressurization commands 1102, adjusting the pressure within the actuator to decrease it to the lowered target pressure 1104.

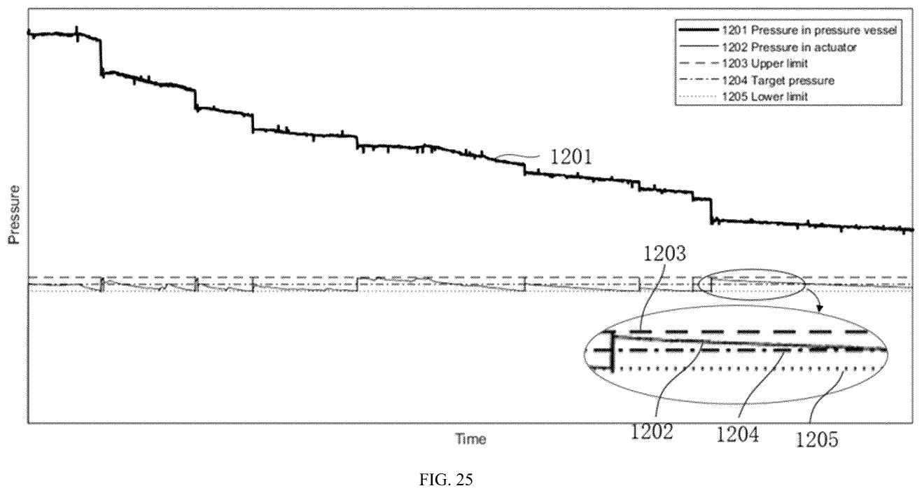

Maintaining pressure within the actuator results in pressure fluctuation curves and corresponding pressure changes within the pressure vessel, as shown in FIG. 25. The control system maintains the fluctuation of the pressure within the actuator 1202 between its upper limit 1203 and its lower limit 1205. When the pressure exceeds the target upper limit 1203, the actuator is depressurized, and when it falls below the target lower limit 1205, the actuator is pressurized to ensure it fluctuates within the target pressure range 1204. When the actuator is pressurized, gas pressure is transferred from the pressure vessel to the actuator, resulting in pressure fluctuation within the pressure vessel 1201. However, by ensuring that the pressure within the pressure vessel is higher than the upper limit of the actuator pressure 1202, the actuator pressure can quickly reach the target value.

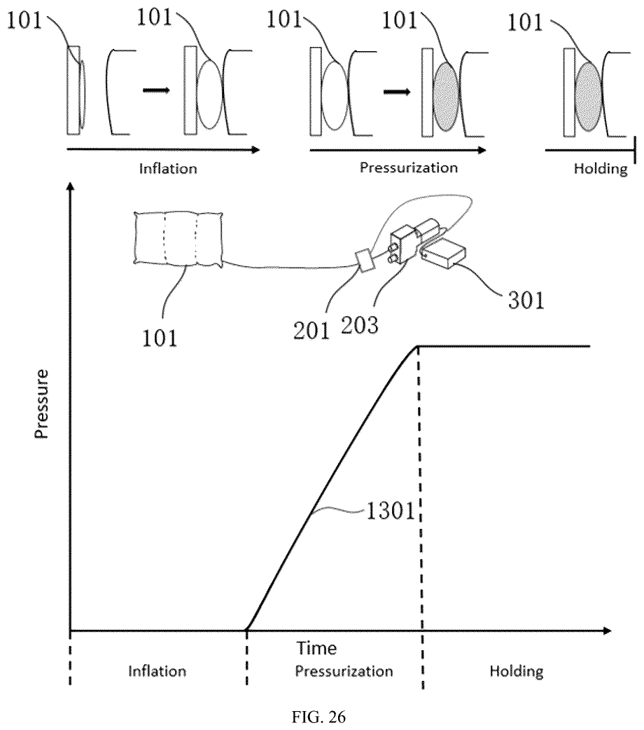

FIG. 26 illustrates the pressure adjustment curve of the pressure regulation loop without a pressure vessel. During the initial pressurization, the air pump 203 and air valve 201 are opened. Since the air pump is not pre-pressurized, the pressure within the actuator 1301 gradually increases from zero, resulting in a relatively long buffering period. When the target pressure is reached, the control system 301 stops the air pump 203 and closes the air valve 201. Therefore, there is a relatively long buffering and pressurization period for actuator pressurization, eventually reaching the target pressure and entering the maintenance phase.

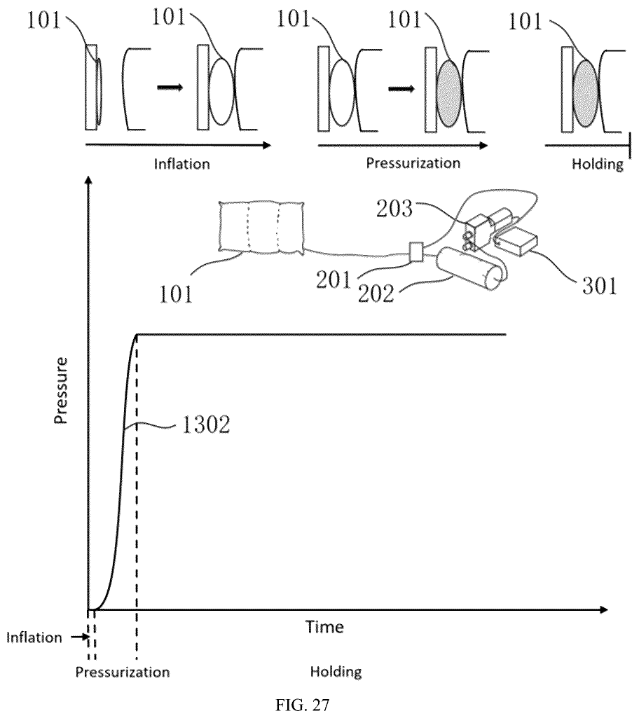

In contrast, the pressure adjustment curve for the actuator 101 with a pressure vessel 202, as shown in FIG. 27, demonstrates that since the pressure vessel 202 is pre-pressurized to a value higher than the required pressure for the actuator, rapid pressurization of the actuator 1302 can be achieved in conjunction with the air pump 203, followed by shutting down the air pump 203 and air valve 201 upon reaching the specified pressure. This control scheme significantly reduces the buffering and pressurization time required for actuator pressurization in MRI scanning scenarios, enhancing diagnostic and therapeutic efficiency, improving the utilization efficiency of MRI scanners 401, and reducing user costs.

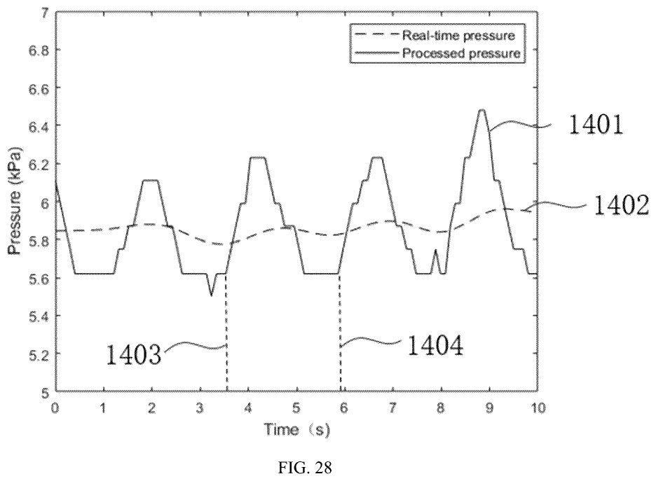

FIG. 28 depicts an example curve of the actual pressure fluctuation of the actuator due to external load changes during the usage process of the MRI-compatible fast dynamic support control system, along with its smoothed handling. The real-time curve 1401 represents real-time values obtained by sensors in the control system, while the dynamic smoothed curve 1402 is obtained by dynamically averaging real-time values obtained by sensors over a certain period. The length of this period is generally determined based on the user's respiratory cycle start 1403 and end 1404 or based on external load change cycles. Real-time value judgment helps avoid excessive pressure application, while dynamic smoothed pressure helps smooth fluctuations caused by breathing or movements, thereby assisting the system in implementing more accurate control.

The above descriptions, in conjunction with the corresponding drawings, illustrate multiple embodiments of the MRI-compatible dynamic support method disclosed herein. The present invention aims to provide a new method for the rehabilitation treatment of relatively stiff joints. However, the present invention is not limited to the specific embodiments described above, which are merely illustrative and not restrictive. Those skilled in the art, guided by the teaching of the present invention, may devise numerous variations within the scope of the invention's purpose and the protected scope of the claims without departing from them.

In summary, the present invention provides an MRI-compatible dynamic composite pressure support control system that offers rapid, visible, and adjustable support capabilities for various orthopedic treatments, particularly in spinal joint orthopedics and stiff joints. By utilizing MRI-visible composite pressure actuators and a supporting frame, coupled with an external pressure control system, the invention enables real-time monitoring and adjustment of orthopedic forces, optimizing treatment efficacy and patient comfort.

While specific embodiments of the invention have been described in detail, various modifications and substitutions may be made thereto without departing from the spirit and scope of the invention. Accordingly, the present invention is not limited to the specific embodiments described herein but is defined by the appended claims and their equivalents.

### Exemplary Embodiments

Embodiment 1. A magnetic resonance imaging (MRI) compatible rapid dynamic support control system, comprising:
at least one dynamic composite pressure support device configured to capture images of MRI scanning; and
a pressure adjustment device coupled with the at least one dynamic composite pressure support device, configured for generating sufficient corrective pressure to apply on a test subject within a certain period, rapidly changing posture of relevant stiff joints, adjusting support configuration and contact deformation of the at least one dynamic composite pressure support device based on rapid MRI scan results, achieving rapid adjustment and correction of stiff joint posture for the test subject, and ensuring dynamic stability of the support force received by the test subject on a stiff joint including cervical, thoracic, and lumbar joints, as well as on a trunk, upper limb, lower limb, neck, or head of the test subject.

Embodiment 2. The MRI-compatible rapid dynamic support control system according to embodiment 1, wherein the at least one dynamic composite pressure support device comprises at least one composite pressure actuator that is visible under MRI scanning, and an MRI-compatible brace configured to cooperate with the at least one composite pressure actuator to provide corrective support force.

Embodiment 3. The MRI-compatible rapid dynamic support control system according to embodiment 2, wherein the at least one composite pressure actuator visible under MRI scanning comprises multiple layers of materials including a sealing layer and an MRI-visible layer.

Embodiment 4. The MRI-compatible rapid dynamic support control system according to embodiment 3, wherein the MRI-visible layer of the at least one composite pressure actuator comprises a material visible under MRI imaging composite on the at least one composite pressure actuator, wherein the material comprises hydrogen atoms such that deformation of the at least one composite pressure actuator is imaged by the MRI, and by controlling a concentration of the hydrogen atoms in the material, a determination of the support effect is obtained without causing artifact in the MRI image.

Embodiment 5. The MRI-compatible rapid dynamic support control system according to embodiment 1, wherein the pressure adjustment device comprises a pressure adjustment unit and circuit wherein the pressure adjustment unit is configured for increasing gas pressure to a pressure vessel by an air pump for pre-pressurization to a higher pressure, thereby shortening inflation time and achieving rapid pressure distribution, allowing at least one composite pressure actuator to cooperate with rapid MRI scanning for adjustment to quickly reach a target pressure; wherein the circuit comprises pipes and pressure transmission, wherein distribution in the pipes is controlled by air valves, and by controlling the air pump and the air valves, pressure adjustment of the at least one composite pressure actuator of the at least one composite pressure support device is achieved.

Embodiment 6. The MRI-compatible rapid dynamic support control system according to embodiment 1, wherein a pressure adjustment loop in the pressure adjustment device is controlled by a control system, the control system being configured to monitor pressure distribution of multiple composite pressure actuators in real-time through pressure sensor detection, and to adjust the pressure adjustment loop according to system setting.

Embodiment 7. The MRI-compatible rapid dynamic support control system according to embodiment 1, wherein during MRI scanning, only the at least one composite pressure support device is placed in a scanning room with an MRI scanner, wherein other devices are placed outside the scanning room, wherein a pressure adjustment loop is connected to the at least one composite pressure support device through a pipe made of MRI-compatible material and wherein a control system is configured to release gas pressure in a pressure vessel to regulate gas pressure, achieving fast pressure adjustment within 1 minute.

Embodiment 8. The MRI-compatible rapid dynamic support control system according to embodiment 2, wherein a shape of the at least one composite pressure actuator is configured to change according to different positions, and different shapes under different pressures are changed to ensure sufficient support force while uniformly distributing pressure on a contact surface with a stiff joint of the test subject, including joints of the trunk, upper limbs, lower limbs, neck, and head; wherein pressure, stretching force, or bending force is applied to the joint.

Embodiment 9. The MRI-compatible rapid dynamic support control system according to embodiment 3, wherein the sealing layer of the composite pressure actuator is configured to withstand gas pressure, thereby providing a pressure required for support or correction and ensuring the support configuration of the at least one composite pressure actuator.

Embodiment 10. The MRI-compatible rapid dynamic support control system according to embodiment 3, wherein an exterior of the at least one composite pressure actuator is wrapped with fabric to enhance comfort at the support location and separate the imaging layer inside the at least one composite pressure actuator from surfaces of the test subject.

Embodiment 11. The MRI-compatible rapid dynamic support control system according to embodiment 1, wherein the control system is configured to track pressure distribution in real-time through control methods, control pumps and valves in the pressure adjustment system to maintain dynamic balance of required pressure, and if the dynamic or real-time pressure of the at least one composite pressure actuator exceeds a set value, the at least one composite pressure actuator is depressurized, while if the pressure of the at least one composite pressure actuator is lower than a set value, the at least one composite pressure actuator is pressurized.

Embodiment 12. The MRI-compatible rapid dynamic support control system according to embodiment 11, wherein the control system comprises a controller and its accompanying circuit, the accompanying circuit includes a pressure sensor, an electromagnetic valve control loop, and a pump control loop, and wherein the controller is configured to obtain values of gas pressure inside the pressure vessel and the at least one composite pressure actuator through the pressure sensor, to change pressure distribution through control of the electromagnetic valve control loop, and to control operation of the electromagnetic pump to pressurize or depressurize the system.

Embodiment 13. The MRI-compatible rapid dynamic support control system according to embodiment 11, wherein the controller of the control system is connected to an external computing device, by wire or wirelessly, and wherein the controller is configured to receive instructions from the external computing device to adjust pressure control strategy parameters and target pressure parameters.

Embodiment 14. The MRI-compatible rapid dynamic support control system according to embodiment 2, wherein the MRI-compatible brace is made of soft materials including pure cotton textiles, or hard materials including plastics, or other MRI-compatible materials.

Embodiment 15. The MRI-compatible rapid dynamic support control system according to embodiment 2, wherein a shape of the MRI-compatible brace is changed according to a different target body segment including the trunk, upper limbs, lower limbs, neck, and head of the test subject.

The invention claimed is:

1. A magnetic resonance imaging (MRI) compatible rapid dynamic support control system, comprising:
at least one dynamic composite pressure support device configured to capture images of MRI scanning; and
a pressure adjustment device coupled with the at least one dynamic composite pressure support device, configured for generating sufficient corrective pressure to apply on a test subject within a certain period, rapidly changing posture of relevant stiff joints, adjusting support configuration and contact deformation of the at least one dynamic composite pressure support device based on rapid MRI scan results, achieving rapid adjustment and correction of stiff joint posture for the test subject, and ensuring dynamic stability of the support force received by the test subject on a stiff joint including cervical, thoracic, and lumbar joints, as well as on a trunk, upper limb, lower limb, neck, or head of the test subject.

2. The MRI-compatible rapid dynamic support control system according to claim 1, wherein the at least one dynamic composite pressure support device comprises at least one composite pressure actuator that is visible under MRI scanning, and an MRI-compatible brace configured to cooperate with the at least one composite pressure actuator to provide corrective support force.

3. The MRI-compatible rapid dynamic support control system according to claim 2, wherein the at least one composite pressure actuator visible under MRI scanning comprises multiple layers of materials including a sealing layer and an MRI-visible layer.

4. The MRI-compatible rapid dynamic support control system according to claim 3, wherein the MRI-visible layer of the at least one composite pressure actuator comprises a material visible under MRI imaging composite on the at least one composite pressure actuator, wherein the material comprises hydrogen atoms such that deformation of the at least one composite pressure actuator is imaged by the MRI, and by controlling a concentration of the hydrogen atoms in the material, a determination of the support effect is obtained without causing artifact in the MRI image.

5. The MRI-compatible rapid dynamic support control system according to claim 1, wherein the pressure adjustment device comprises a pressure adjustment unit and circuit wherein the pressure adjustment unit is configured for increasing gas pressure to a pressure vessel by an air pump for pre-pressurization to a higher pressure, thereby shortening inflation time and achieving rapid pressure distribution, allowing at least one composite pressure actuator to cooperate with rapid MRI scanning for adjustment to quickly reach a target pressure; wherein the circuit comprises pipes and pressure transmission, wherein distribution in the pipes is controlled by air valves, and by controlling the air pump and the air valves, pressure adjustment of the at least one composite pressure actuator of the at least one composite pressure support device is achieved.

6. The MRI-compatible rapid dynamic support control system according to claim 1, wherein a pressure adjustment loop in the pressure adjustment device is controlled by a control system, the control system being configured to monitor pressure distribution of multiple composite pressure actuators in real-time through pressure sensor detection, and to adjust the pressure adjustment loop according to system setting.

7. The MRI-compatible rapid dynamic support control system according to claim 1, wherein during MRI scanning, only the at least one composite pressure support device is placed in a scanning room with an MRI scanner, wherein other devices are placed outside the scanning room, wherein a pressure adjustment loop is connected to the at least one composite pressure support device through a pipe made of MRI-compatible material and wherein a control system is configured to release gas pressure in a pressure vessel to regulate gas pressure, achieving fast pressure adjustment within 1 minute.

8. The MRI-compatible rapid dynamic support control system according to claim 2, wherein a shape of the at least one composite pressure actuator is configured to change according to different positions, and different shapes under different pressures are changed to ensure sufficient support force while uniformly distributing pressure on a contact surface with a stiff joint of the test subject, including joints of the trunk, upper limbs, lower limbs, neck, and head; wherein pressure, stretching force, or bending force is applied to the joint.

9. The MRI-compatible rapid dynamic support control system according to claim 3, wherein the sealing layer of the composite pressure actuator is configured to withstand gas pressure, thereby providing a pressure required for support or correction and ensuring the support configuration of the at least one composite pressure actuator.

10. The MRI-compatible rapid dynamic support control system according to claim 3, wherein an exterior of the at least one composite pressure actuator is wrapped with fabric to enhance comfort at the support location and separate the imaging layer inside the at least one composite pressure actuator from surfaces of the test subject.

11. The MRI-compatible rapid dynamic support control system according to claim 1, wherein the control system is configured to track pressure distribution in real-time through control methods, control pumps and valves in the pressure adjustment system to maintain dynamic balance of required pressure, and if the dynamic or real-time pressure of the at least one composite pressure actuator exceeds a set value, the at least one composite pressure actuator is depressurized, while if the pressure of the at least one composite pressure actuator is lower than a set value, the at least one composite pressure actuator is pressurized.

12. The MRI-compatible rapid dynamic support control system according to claim 11, wherein the control system comprises a controller and its accompanying circuit, the accompanying circuit includes a pressure sensor, an electromagnetic valve control loop, and a pump control loop, and wherein the controller is configured to obtain values of gas pressure inside the pressure vessel and the at least one composite pressure actuator through the pressure sensor, to change pressure distribution through control of the electromagnetic valve control loop, and to control operation of the electromagnetic pump to pressurize or depressurize the system.

13. The MRI-compatible rapid dynamic support control system according to claim 11, wherein the controller of the control system is connected to an external computing device, by wire or wirelessly, and wherein the controller is configured to receive instructions from the external computing device to adjust pressure control strategy parameters and target pressure parameters.

14. The MRI-compatible rapid dynamic support control system according to claim 2, wherein the MRI-compatible brace is made of soft materials including pure cotton textiles, or hard materials including plastics, or other MRI-compatible materials.

15. The MRI-compatible rapid dynamic support control system according to claim 2, wherein a shape of the MRI-compatible brace is changed according to a different target body segment including the trunk, upper limbs, lower limbs, neck, and head of the test subject.
